(19) United States
(12) Patent Application Publication
(10) Pub. No.: US 2011/0218606 A1
(43) Pub. Date: Sep. 8, 2011

(54) Methods for Stabilizing Femoral Vessels

(75) Inventors: Mark J. Dolan, Santa Rosa, CA (US); Julie Trudel, Santa Rosa, CA (US); Stefan Tunev, Santa Rosa, CA (US)

(73) Assignee: Medtronic Vascular, Inc., Santa Rosa, CA (US)

(21) Appl. No.: 12/715,550

(22) Filed: Mar. 2, 2010

(51) Int. Cl. A61M 5/178; A61F 2/82; A61L 27/24

(52) U.S. Cl. 623/1.1; 604/507

(57) ABSTRACT

The invention provides methods for stabilizing the femoral artery.

### BACKGROUND

The adductor canal is a tunnel-like area defined by the membranes separating the thigh muscles. It is located in the middle third of the thigh. The adductor canal contains the femoral artery, the femoral vein and the femoral nerve. The adductor hiatus region is located in a gap between the adductor magnus muscle and the femur and allows the passage of the femoral vessels from the posterior thigh to the popliteal fossa. The adductor hiatus region is the termination of the adductor canal and lies about 2 inches superior to the knee. The physical constraints imposed by this particular anatomy on vessels as they move in these regions can cause stress, e.g., for the artery and any associated medical devices, and can this cause loading and fatigue problems for the medical device. This area is a common area for the failure of the vessels and medical devices, such as stents. Methods are needed that can alleviate the stress on the vessels and any associated medical device.

### SUMMARY OF CERTAIN EMBODIMENTS OF THE INVENTION

Certain embodiments of the present invention provide methods for stabilizing a femoral vessel, comprising delivering an amount a material in a generally fluid state to an area around the femoral vessel, wherein following delivery, the generally fluid material increases in strength sufficient to stabilize the femoral vessel. The femoral vessel may be located in a mammal, such as a human male or female, or in a non-human mammal.

In certain embodiments, the material is delivered to an area around the femoral vessel in the adductor canal region.

In certain embodiments, the material is delivered to an area around the femoral vessel in the adductor hiatus region.

In certain embodiments, the femoral vessel is the femoral artery.

In certain embodiments, the femoral vessel is the femoral vein.

In certain embodiments, the femoral vessel comprises a medical device.

In certain embodiments, the femoral vessel comprises a stent.

In certain embodiments, the material is delivered to an area around the femoral vessel to fully surround the vessel around its circumference.

In certain embodiments, the material is delivered to an area around the femoral vessel from about 1 cm to about 10 cm longitudinally along the vessel.

In certain embodiments, the material comprises collagen.

In certain embodiments, the collagen is type I or III collagen.

In certain embodiments, the material comprises poly(lactic-co-glycolic acid) (PLGA).

In certain embodiments, the material comprises an alginate hydrogel.

In certain embodiments, the material comprises a photocurable hydrogel.

In certain embodiments, the material is a biodegradable material.

In certain embodiments, the material is a non-biodegradable material.

In certain embodiments, the material is delivered using direct injection.

In certain embodiments, the material is delivered percutaneously, comprising puncturing the vessel wall and delivering the material around the vessel.

### DETAILED DESCRIPTION

Peripheral vessel movement, including bending, torsion, compression and tension, can be the source of problematic conditions for blood vessels. This can be a particular problem for a vessel, such as an artery, that has a medical devices (e.g., a stent) associated with the vessel. While the vessel may in certain cases be flexible enough to accommodate for such physical forces, a medical device associated with the vessel can decrease the ability of the vessel to accommodate such forces and can lead to problems, such as device fatigue and failure, and damage to the vessel. A method for stabilizing the vessel in a manner that also allows for function would reduce the stress induced by a medical device. Accordingly, such methods are described herein.

The methods described herein, in certain embodiments, involve the delivery of material to an area that surrounds, at least in part, a vessel. After delivery, the material functions to stabilize and support the vessel by reducing physical forces on the vessel. For example, a generally fluid material can be delivered to surround the femoral artery in the adductor hiatus area. After delivery, the generally fluid material increases in hardness and strength to form a “soft cast” around the artery. This method can be useful for treating the femoral artery alone, and can also be useful for treating the femoral artery when a medical device, such as a stent, is associated with the femoral artery. Following delivery of the material, the femoral artery, and any associated medical device, is stabilized, thereby reducing the physical forces applied to the femoral artery.

As used herein, to “stabilize” a vessel means to distribute load over a relatively larger surface area, as opposed to distributing load over a small portion of the anatomy of a vessel. This would modify the distribution of forces applied to the vessel as compared to the force distribution that would have been applied without the stabilization. This stabilization can include decreasing the local amount of physical force caused by bending, torsion, compression and tension of the vessel, and modifying load distribution imposed on a vessel by a medical device associated with the vessel, such as a stent.

The “strength” of a material can be described as its ability to withstand an applied stress. A material's microstructure can affect its strength. Strength is considered in terms of, e.g., compressive strength, tensile strength, and shear strength. The effects of dynamic loading are an important practical part of the strength of materials, especially in relation to the problem of fatigue. Thus, a material has an increased “strength” when the material is more able to withstand an applied stress.

### Treatment Areas

The adductor canal is a tunnel-like area defined by the membranes separating the thigh muscles from each other. It is located in the middle third of the thigh. The adductor canal contains the femoral artery, the femoral vein and the femoral nerve. Most femoral artery occlusions result from atherosclerotic disease progression in the adductor canal region. Moreover, femoral artery atherosclerotic disease progresses to occlusion at a more rapid rate in this region. This may be the result of mechanical stress or mechanical restriction of the artery, thereby preventing compensatory enlargement of the artery as atherosclerosis develops. Further, the movement of the artery in the adductor canal can create loading and fatigue problems, e.g., for medical devices. In certain embodiments, the methods described herein include delivering the material so that the material surrounds, at least in part, the femoral artery or the femoral vein in the adductor canal.

The adductor hiatus region is defined as a gap between the adductor magnus muscle and the femur and allows the passage of the femoral vessels from the posterior thigh to the popliteal fossa. It is the termination of the adductor canal and lies about 2 inches superior to the knee. In certain embodiments, the methods described herein include delivering the material so that the material surrounds, at least in part, the artery or the vein in the adductor hiatus region.

While in certain embodiments these methods are used in the area of the femoral vessels (e.g., the femoral artery, femoral vein, including the popliteal artery; see Netter, Atlas of Human Anatomy, 4^{th} Edition (2006) at, e.g., Plates 500 and 512), this method also find use in other locations in the body. For example, such methods may be utilized to deliver the materials into the cranial cavity to treat (e.g., contain and decrease) bleeding in the meninges caused by, e.g., a subdural haematoma. These methods may also be applied to other vessels, for example, vessels located in areas that are subject to physical stress caused by, e.g., relatively higher levels of bending and in areas where the canal in which the vessel is located is subject to constriction. Other areas in which these methods may be used include areas that are relatively difficult to access surgically.

In certain embodiments, the materials are delivered to an area around a vessel to stabilize an area of the vessel from about 1-10 cm long (e.g., about 1 cm, 2 cm, 3 cm, 4 cm, 5 cm, 6 cm, 7 cm, 8 cm, 9 cm, 10 cm). In certain embodiments the material fully surrounds the vessel. In certain embodiments the material does not fully surround the vessel.

In certain embodiments, the methods described herein can also be used to treat peripheral vascular disease (PVD). PVD, also known as peripheral artery disease (PAD) or peripheral artery occlusive disease (PAOD), includes diseases caused by the obstruction of large arteries in the arms and legs. PVD can result from, e.g., atherosclerosis, inflammatory processes leading to stenosis, an embolism or thrombus formation, and can cause acute or chronic ischemia due to lack of blood supply, typically to the legs.

### Materials

Different types of materials may be selected by the art worker, depending, e.g., upon the amount of stabilization of the vessel desired. The selection of material may be based at least in part, upon the ability of the material to minimize movements of the vessel while permitting limb movement, the durability of the material, the migration properties of the material, and ease of delivery. In certain embodiments, the material is a biodegradable material. In certain embodiments, the material is a non-biodegradable material.

In certain embodiments, the material comprise substances found naturally in the human body, such as collagen, e.g., collagen type 3. Another type of material that may be used is an inorganic material such as poly(lactic-co-glycolic acid) (PLGA). Another type of material that could be used is organic material such as an alginate hydrogel.

In certain embodiments, the materials comprise or consists essentially of a photocurable hydrogel, which may be degradable or non-degradable. For a non degradable hydrogel, PEG diacrylate may be used (see, e.g., An et al., Journal of Controlled Release, 64, 205-215 (2000). For a degradable hydrogel, for example, a lactic acid ester group can be inserted into PEG before adding acrylate end caps (see, e.g., Lyman et al., Biomaterials, 17, 359-364 (1996). Eosin-Y may be used to initiate photocrosslinking via electron generation upon light activation at the proper wavelength. A light source can be used to transform the fluid macromer precursor into crosslinked hydrogel. For example, a solid state green laser system set at, e.g., 532 nm can be used. The light can be delivered to the target area using a light diffuser, e.g., encased in a catheter. In certain embodiments, a light diffuser can be positioned inside the vessel and the wattage of the laser adjusted to cause crosslinking of the material outside the vessel. Such a light diffuser can be, in certain embodiments, up to about 10 cm in length (e.g., about 1, 2, 3, 4, 5, 6, 7, 8, 9 or 10 cm in length). The material may be delivered in combination with a wire mesh structure, e.g., a nitinol wire mesh structure.

### Delivery of Materials

The material in certain embodiments is delivered through direct injection. Guidance, such as ultrasound guidance, may be used to assist in the delivery of the material. Another method for delivery is percutaneous to puncture the vessel wall and to inject the material around the vessel. Such methods may utilize a catheter. In other embodiments, a dual layer covered stent may be used, using the void between the layers and external elution holes to release the material (see, e.g., U.S. Pat. No. 7,377,937).

In one embodiment, the vessel (e.g., artery) wall can be percutaneously punctured and the surrounding area filled with a material to an extent sufficient to stabilize the vessel. The material will fill the area, and in some embodiments may fully surround the vessel. In some embodiments, the material is delivered in one administration, e.g., injection. In other embodiments, the material is delivered in multiple administrations, e.g., injections. In other embodiments, the material will not fully surround the vessel, but will fill the area to the extent needed to support the vessel more than in the absence of the material. In some embodiments, this method can be used to stabilize the vessel when inner luminal scaffold, such as a stent, is present.

As used herein, the percutaneous method involves accessing a blood vessel via a needle puncture followed by an introducer device that will serve as a port for catheter access to the target location. The introducer site can be the femoral artery or vein.

The invention will now be illustrated by the following non-limiting Example.

### EXAMPLE 1

A patient, who has been diagnosed as needing placement of a stent in the femoral artery, will also receive a soft cast to stabilize the femoral artery. The soft cast will fully surround the femoral artery around its circumference and will extend longitudinally past the location of each end of the stent. Briefly, an amount a photocurable hydrogel will be delivered percutaneously to the area surrounding the femoral artery so that the soft cast will extend about 1 mm to about 2 mm longitudinally past the location for each end of the stent. Following introduction of the stent, the photocurable hydrogel will be cured, and the hydrogel will increase in strength sufficient to stabilize the femoral artery.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for stabilizing a femoral vessel, comprising delivering an amount a material in a generally fluid state to an area around the femoral vessel, wherein following delivery, the generally fluid material increases in strength sufficient to stabilize the femoral vessel.

2. The method of claim 1, wherein the material is delivered to an area around the femoral vessel in the adductor canal region.

3. The method of claim 1, wherein the material is delivered to an area around the femoral vessel in the adductor hiatus region.

4. The method of claim 1, wherein the femoral vessel is the femoral artery.

5. The method of claim 1, wherein the femoral vessel is the femoral vein.

6. The method of claim 1, wherein the femoral vessel comprises a medical device.

7. The method of claim 1, wherein the femoral vessel comprises a stent.

8. The method of claim 1, wherein the material is delivered to an area around the femoral vessel to fully surround the vessel around its circumference.

9. The method of claim 1, wherein the material is delivered to an area around the femoral vessel from about 1 cm to about 10 cm longitudinally along the vessel.

10. The method of claim 1, wherein the material comprises collagen.

11. The method of claim 10, wherein the collagen is type I or III collagen.

12. The method of claim 1, wherein the material comprises poly(lactic-co-glycolic acid) (PLGA).

13. The method of claim 1, wherein the material comprises an alginate hydrogel.

14. The method of claim 1, wherein the material comprises a photocurable hydrogel

15. The method of claim 1, wherein the material is a biodegradable material.

16. The method of claim 1, wherein the material is a non-biodegradable material.

17. The method of claim 1, wherein the material is delivered using direct injection.

18. The method of claim 1, wherein the material is delivered percutaneously, comprising puncturing the vessel wall and delivering the material around the vessel.
